(19) United States
(12) Patent Application Publication
(10) Pub. No.: US 2005/0037472 A1
(43) Pub. Date: Feb. 17, 2005

(54) Method for recovering and purifying polyglutamic acid

(76) Inventors: Ing-Lung Shih, Yuanlin Township (TW); I-Chung Fan, Toufen Township (TW); Ming-Hao Shen, Chiayi City (TW)

Correspondence Address: SUPREME PATENT SERVICES, POST OFFICE BOX 2339, SARATOGA, CA, 95070, US

(21) Appl. No.: 10/641,880

(22) Filed: Aug. 16, 2003

(51) Int. Cl. C12P 7/62; C12N 1/20; C08G 63/78

(52) U.S. Cl. 435/135; 435/252.31; 528/274

(57) ABSTRACT

A method for recovering and purifying polyglutamic acid efficiently is disclosed, which method includes the steps of adjusting pH of a solution containing γ-PGA to neutral or slightly acidic range and filtering the solution through a plurality of filtration membranes with various molecular weight cutoffs to recover γ-PGA. Advantages of the method includes using none or minimum of organic solvent, reducing consumption of filtration membranes, reducing water volume for dilution, shortening operation time, high productivity, and obtaining various γ-PGA products with different range of molecular weight.

### BACKGROUND OF THE INVENTION

1. Field of the Invention

The present invention relates to a method for recovering and purifying poly-γ-glutamic acid (herein after is referred to as γ-PGA), more particularly to a process for recovering and purifying γ-PGA from culture broth of microorganisms.

2. Prior Arts

γ-PGA is an unusual anionic, naturally occurring homo-polyamide that is made of D- and L-glutamic acid units connected by amide linkages between α-amino and γ-carboxylic acid groups. It is a highly viscous material produced extracellularly by a variety of Bacillus species. Being a water soluble, edible, biodegradable and non-toxic material, γ-PGA has become an attractive investigated target to many researchers in different fields recently. γ-PGA and its derivates are widely applicable to a broad range of industrial fields such as food, cosmetics, medicine and water treatment. Applications for foodstuff, γ-PGA can be as a thickener of food (or drink), an antifreezing agent, a bitterness-relieving agent, a stabilizer in ice cream, and as an additive in the preparation of starch foods (mainly bakery products and noodles) for avoiding aging, enhancing food texture and maintaining food shape. Applications to cosmetics, γ-PGA can be as a humectant. Applications to medicine, γ-PGA can be as a drug carrier, a haemostatic agents or a surgical adhesive. For water treatment, γ-PGA can be as a flocculant for treating waste water, an absorbent for binding heavy metal or radionuclides. For other applications, γ-PGA derivatives act as an excellent hydrogel and water absorbent that has potential use as sanitary materials, water reservoirs in agriculture, and hydrogel implants for drug-release. γ-PGA derivatives can also form biodegradable fibers and films that can replace currently used non-biodegradable polymers, they can act as excellent thermoplastic that can be processed into fibers or membranes with excellent strength, transparency and elasticity. Because of its wide applications, excellent characteristics and non-toxic toward humans and the environment, countries in Europe and America, Japan and Korea are paying much attention for the production of γ-PGA.

Presently, polymerization of glutamic acid by fermentation of microorganisms is a major process to produce γ-PGA. In a polymerized process by fermentation, the viscosity of culture broth is highly increased with production of the γ-PGA polymer. It is necessary to carry out centrifugation at high speed to remove microorganisms after fermentation. For example, the centrifuged speed is 12,000 to 20,000 rpm for a culture broth with neutral pH value. Hereof, an expensive instrument capable of operating at high speed is needed. Moreover, a great amount of power and electricity is consumed to remove microorganisms from culture broth. Besides, it is difficult to recover and purify the product of γ-PGA due to the high viscosity in fermented culture broth.

For recovering and purifying γ-PGA from cell-free culture broth, the precipitation method is a widely used process. After the removal of microorganisms from fermented broth, γ-PGA was precipitated from cell-free broth with a large volume of organic solvent. The crude precipitated product is repeatedly solubilized and dialyzed against deionized water. The dialyzed solution is centrifuged and the supernatant is lyophilized to afford γ-PGA of high purity. A disadvantage for the method is the usage of large volumes of organic solvent to precipitate product in the process. In general, the volume ratio of organic solvent to fermented broth is 4:1. Even such a large volume is not capable of precipitating γ-PGA out from culture broth completely. A loss of γ-PGA about 5 to 15% occurs in a general operated process. Besides, the usage of large volumes of solvent may bring environmental problem and be harmful to operator carrying out the process.

To solve the aforementioned disadvantage, alternative method for recovering and purifying γ-PGA is developed. It firstly removes low molecular weight materials by a filtration membrane, and then carried out processes like alcohol precipitation, crystallization at low temperature or vacuum drying under heating to recover γ-PGA. To elevate the effect of recovery and purification, it is necessary to adjust the pH value of fermented broth and select an adequate filtration membrane. When the pH value of fermented broth is adjusted to acidic range (pH 1 to 3), γ-PGA possess a helical (linear) conformation resulting from the reduction of the hydrodynamic radius that enhance the permeability of γ-PGA in membrane and leads to a serious loss of γ-PGA in filtration under pressure. Moreover, the filtration membrane is easily damaged by acidic solution, and it leads to large consumptions of filtration membrane. When the pH value of fermented broth is adjusted to pH 5 to 7, γ-PGA becomes highly viscous and it behaves like a random coil, a conformation that leads to high retention and elevate recovery of γ-PGA, but the membrane is easily blocked by the resulting high viscous fermented broth. To decrease viscosity of the broth, dilution with large volume of water is employed, but it increases the operation time and the cost.

In summary, there are strong reasons for developing a method to recover and purify γ-PGA efficiently and use none or minimum of any organic solvent.

### SUMMARY OF THE INVENTION

A primary object of the present invention is to provide a method for recovering and purifying γ-PGA efficiently. The method adjusts the pH value of a solution containing γ-PGA to neutral or slightly acid pH, and filters the solution through a plurality of membranes with various molecular weight cutoffs. The method recovers and purifies γ-PGA efficiently, decreases consumptions of filtration membranes, reduces water volume for dilution, shortens the operation time, and use none or minimum of any organic solvent.

Another object of the present invention is to provide a method for recovering and purifying γ-PGA efficiently. Through the method, various γ-PGA products with different range of molecular weight are obtained after the recovering and purifying process.

The process of the present invention is illustrated by a flowchart shown as FIG. 1. Firstly, the pH value of the microbiologically fermented broth containing γ-PGA is adjusted below pH 2. The acidic pH leads to a less viscous broth and decreases negative charges on cell surfaces. At low pH, the cells lost their surface charge are easily aggregated and settle down; the secreted extracellular γ-PGA is more difficult to adhere to the cell surface. The microorganisms can be removed by centrifugation at lower speed. After removing microorganisms, the pH value of the acid broth is adjusted to slightly acidic or neutral range (about pH 5 to 7) with alkaline solution. The broth after adjusting pH is diluted with adequate volume of water (water volume is about 3 to 5 fold based on the broth volume). A plurality of membranes are selected according to the distribution of molecular weight of γ-PGA in fermented broth, and then filtrations through the selected membranes are carried out to recover various γ-PGA products with different range of molecular weight. The present invention selects adequate membranes with various molecular weight cutoffs according to the molecular weight distribution of produced γ-PGA in fermented broth, and filtrations are processed with the membranes in a pore size sequence from large to small to recover and purify γ-PGA, thereby prolonging the life of membranes and obtaining various γ-PGA products with different range of molecular weight.

Advantages of the present invention include: (1)Removal of microorganism can be achieved without centrifugation at high speed, (2)less water is needed for dilution to process filtration, (3)operation time is shortened, (4)γ-PGA is recovered efficiently, (5)consumption or blocking of filtration membranes is reduced to prolong the life of membranes, (6)various γ-PGA products with different range of molecular weight can be obtained, and (7)none or minimum of organic solvents is used.

Having been fully described the present invention, examples illustrating its practice are set forth below. These examples should not, however, be considered to limit the scope of the invention, which is defined by the appended claims.

### BRIEF DESCRIPTION OF THE DRAWINGS

The related drawings in connection with the detailed description of the present invention to be made later are described briefly as follows, in which:

FIG. 1 is a flow chart of the present invention for recovering and purifying γ-PGA.

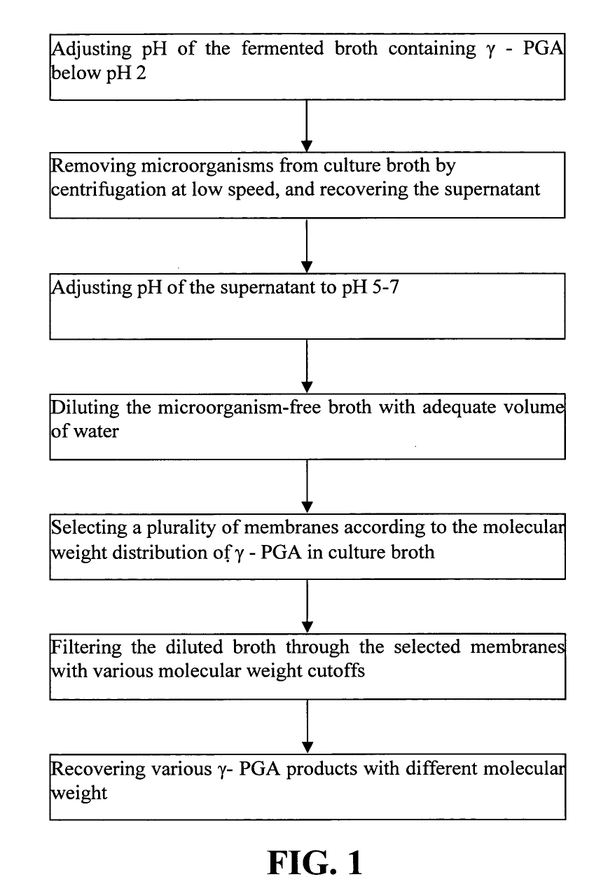

FIG. 2 is a chromatogram of the γ-PGA molecular weight distribution in the fermented broth using Bacillus licheniformis.

FIG. 3 is a chromatogram of amino acid analysis for the γ-PGA obtained by the process of the present invention.

FIG. 4 is a chromatogram of H¹-NMR for the γ-PGA obtained by the process of the present invention.

FIG. 5 is a chromatogram of C¹³-NMR for the γ-PGA obtained by the process of the present invention.

FIG. 6 is a chromatogram of infrared spectrum for the γ-PGA obtained by the process of the present invention.

### DETAILED DESCRIPTION OF THE PREFERRED EMBODIMENT

One embodiment of the present invention is described in the followings. Culturing of Bacillus licheniformis (ATCC 9945) is carried out to produce γ-PGA. After culturing, the pH value of the fermented broth is adjusted below pH 2 and stirred for a period of time. The microorganisms are removed by centrifugation, preferably performed at 4° C. and 6,000 to 9,000 rpm for 20 to 30 minutes. The viscosity of the fermented broth can be lowered, and centrifugation at high speed is not necessary for removing microorganisms. After centrifugation, the supernatant containing γ-PGA can be stored at 4° C. to avoid γ-PGA degradation before a purification process. The debris suspended in the broth can be removed by a membrane of pore size 0.45μ, and the broth can be decolored by charcoal if necessary. The cell-free broth is adjusted to about pH 5 to 7, and diluted with water to precede filtrations, a water volume of 3 to 5 fold based on the volume of broth being preferable for dilution. The diluted broth is filtrated through membranes to recover and purify γ-PGA. According to the γ-PGA molecular weight distribution obtained by molecular weight analysis in broth, the broth firstly passes through a membrane with a molecular weight cutoff more than 500 kD to recover the γ-PGA with higher molecular weight (more than 3,000,000) and then passes through a membrane with a molecular weight cutoff of 10 kD to recover the γ-PGA with lower molecular weight (from tens of thousands to hundreds of thousands). The filtrations can be circulated several times to elevate recovery. The recovered γ-PGA cab be lyophilized, spray dried or reduced pressure dried to form a salt of PGA.

### EXAMPLE 1

### Fermentation of Bacillus licheniformis to Produce γ-PGA

A 7 L of medium containing 65 g /L sodium glutamate, 22 g/L citric acid, 170 g/L glycerin, 7 g/L NH₄Cl, 0.5 g/L MgSO₄.4˜6H₂O, 0.15 g/L MnSO₄.4˜6H₂O, 0.15 g/L CaCl₂.2 H₂O, 0.04 g/L FeCl₃.6H₂O, 0.5 g/L K₂HPO₄.4˜6H₂O is prepared for culture, and the pH value of the medium is adjusted to about pH 6.5.

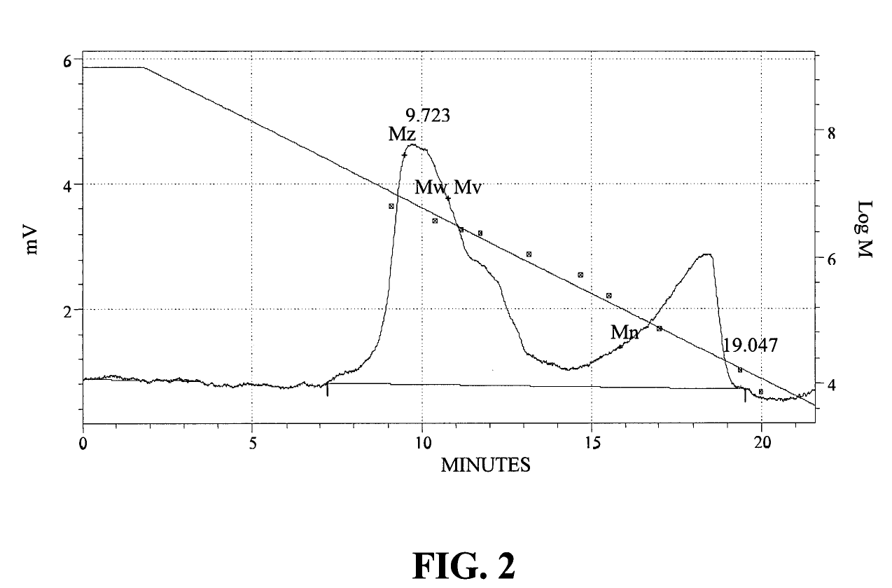

The activated Bacillus licheniformis is inoculated into the medium, cultured in a 10 L of fermentor. Fermentation is performed at 37° C. for 96 hours under a condition of controlled pH 6.5, stirring speed of 200 rpm and aeration rate of 3 vvm. After fermentation, viscosity measurement of the broth indicates a viscosity of 238 cp. Additionally, the molecular weight distribution of γ-PGA in final fermented broth is determined by gel permeation chromatography (GPC) measurement, Mw is 3,688,149, Mn is 156,002 and Mw/Mn is 23.641. The result of GPC measurement is shown as FIG. 2, γ-PGA can be fractionated into two groups; one is high molecular weight (thousands of thousands) and the other is low molecular weight (tens of thousands to hundreds of thousands).

### EXAMPLE 2

### Recovery and Purification of γ-PGA

The final fermented broth obtained in accordance with Example 1 is adjusted to pH 2, stirred for 30 minutes, and centrifuged at 4° C. for 30 minutes to remove microorganisms. The supernatant after centrifugation is adjusted to about pH 7 with 6 N sodium hydroxide, and then diluted by adding four-fold volume of water. According to the molecular weight distribution of γ-PGA in broth, two molecular weight cutoffs of membranes are employed in the filtration process. The diluted broth is processed by the filtration system (Pellicon 2, Millipore), firstly through a membrane of 500 kD molecular weight cutoff and circulated twice to obtain the high molecular weight polymer (thousands of thousands), then the filtrate is passed through a membrane of 10 kD molecular weight cutoff and circulated twice to obtain the low molecular weight polymer (tens of thousands to hundreds of thousands).

The product of γ-PGA obtained from the present invention is characterized by the following analysis:

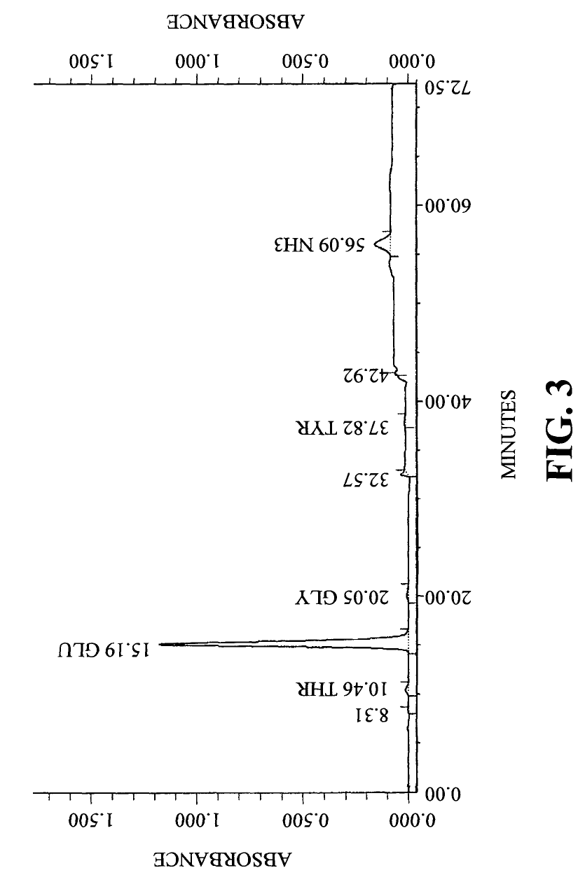

Analysis of amino acid: The obtained γ-PGA is hydrolyzed with 6 N HCl at 110° C. for 24 hours and the hydrolyte is analyzed by Beckman system 6300E equipped with a column filled with Beckman PA-35 resin (0.9×55 cm). The resulting chromatogram is illustrated in FIG. 3.

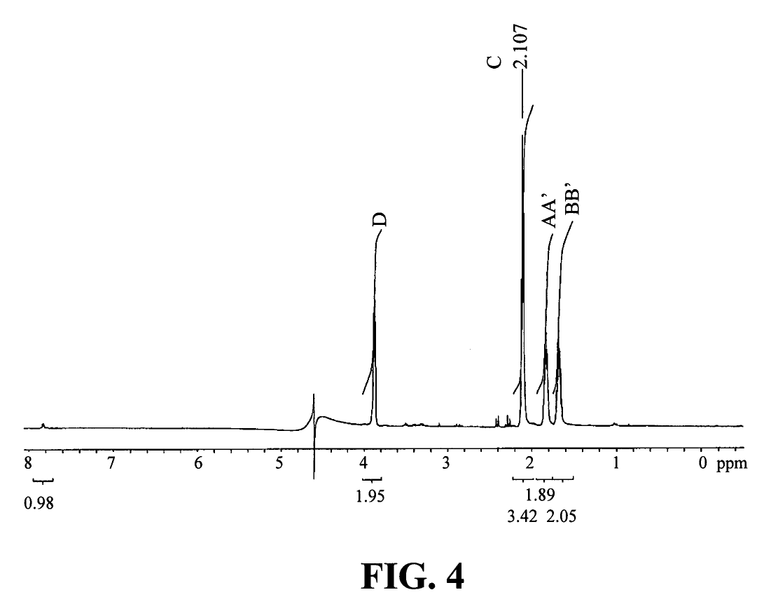

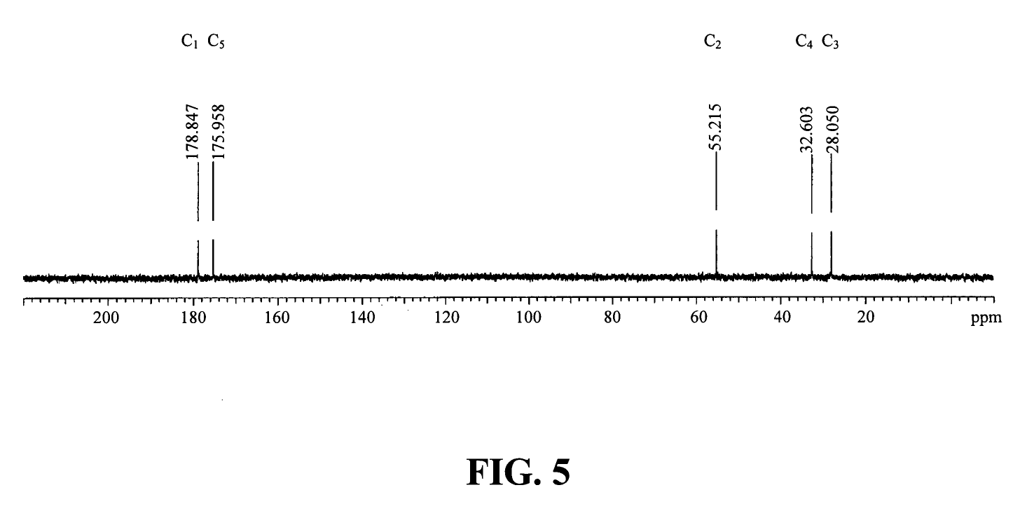

NMR analysis: The obtained γ-PGA is dissolved in D₂O and analyzed by NMR spectrometer (Varian Unity Inova 600 MHz FT NMR). The resulting chromatograms of H¹-NMR and C¹³-NMR are shown in FIG. 4 and 5, respectively. The notes representing the position of hydrogen in the chromatogram of FIG. 4 are in accordance with the following formula: 1.6-1.8 (AA′, BB′, β, 2H); 2.1(C, γ, 2H); 3.8 (D, α, 1H) and 7.8 (N—H). 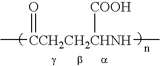
The notes representing the position of carbon in the chromatogram of FIG. 5 are in accordance with the following formula: 178(1); 174(5); 55(2); 33(4) and 28(3).

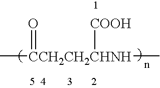

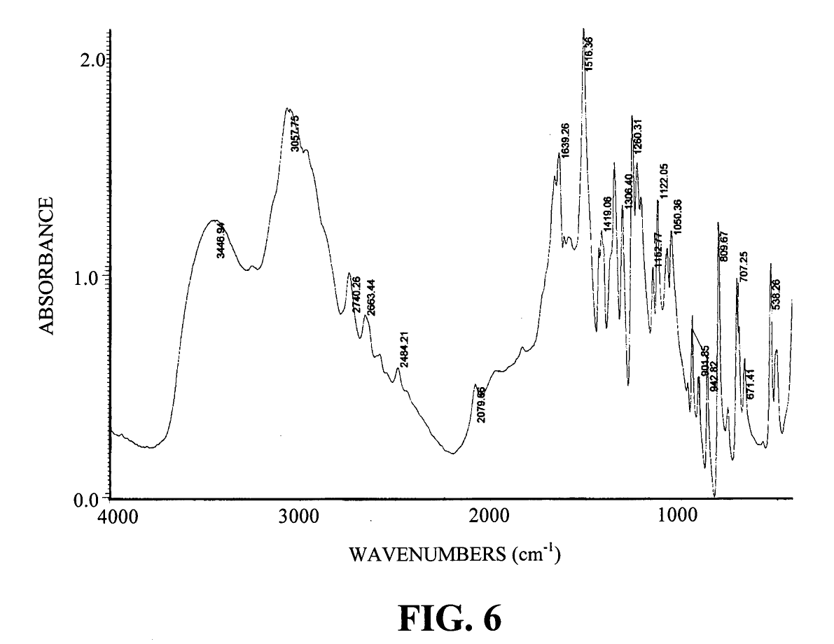

Analysis of infrared spectrum: The obtained γ-PGA is analyzed by infrared spectrophotometers, the resulting spectrum is shown in FIG. 6. 3300-3500 cm⁻¹ is represented as N—H, 3250-3550 cm⁻¹ is represented as OH of hydroxyl group, 2500-3000 cm⁻¹ is represented as hydrogen bond of hydroxyl group, and 1630-1780 cm⁻¹ is represented as C═C.

Recovered γ-PGA is lyophilized to obtain sodium poly glutamate, 57.42 g of sodium poly glutamate is obtained from per litter fermented broth after the process of the present invention.

### EXAMPLE 3

A culture is performed as described in Example 1 except the culture medium contains 50 g /L sodium glutamate, 16 g/L citric acid, 135 g/L glycerin, 7 g/L Urea, 0.5 g/L MgSO₄.7 H₂O, 0.15 g/L MnSO₄.4-6 H₂O, 0.15 g/L CaCl₂.2 H₂O, 0.04 g/L FeCl₃.6H₂O, 0.5 g/L K₂HPO₄.4-6 H₂O. The resulting broth is performed described in Example 2 to recover and purify γ-PGA, 34.65 g of sodium poly glutamate is obtained from per litter fermented broth after the process of the present invention.

### EXAMPLE 4

A culture is performed as described in Example 1 except the culture medium contains 20 g /L sodium glutamate, 12 g/L citric acid, 80 g/L glycerin, 7 g/L NH₄Cl, 0.5 g/L MgSO₄.7 H₂O, 0.15 g/L MnSO₄.4-6 H₂O, 0.15 g/L CaCl₂.2 H₂O 0.04 g/L FeCl₃.6H₂O, 0.5 g/L K₂HPO₄. The resulting broth is performed as described in Example 2 to recover and purify γ-PGA, 19.2 g of sodium poly glutamate is obtained from per litter fermented broth after the process of the present invention.

From the aforementioned examples, the present method for recovering and purifying γ-PGA is applicable to different culture media and high productivity of γ-PGA is always achievable.

In viewing above said, the present invention provides a simple process capable of shortening operation time, recovering γ-PGA efficiently, reducing consumptions of filtration membranes, avoiding blocking of filtration membranes, and obtaining various γ-PGA products with different range of molecular weight with none or minimum use of organic solvent.

1. A method for recovering and purifying γ-PGA from solution, comprising the steps of:
(a) adjusting pH of a solution containing γ-PGA to pH 5-7;
(b) fractionating a plurality of γ-PGA groups in the solution according to a distribution of γ-PGA molecular weight;
(c) selecting a plurality of filtration membranes according to the molecular weight of the γ-PGA groups of step (b);
(d) filtering the solution of step (a) through the selected membranes of step (c) in a sequence of molecular weight cutoff from high to low; and
(e) recovering various γ-PGA products with different range of molecular weight.

2. The method of claim 1, wherein the solution of step (a) is a culture broth of microorganism.

3. The method of claim 2, wherein the microorganism is Bacillus licheniformis.

4. A method for recovering and purifying γ-PGA from culture broth of microorganism, comprising the steps of:
(i) removing microorganisms from a culture broth containing γ-PGA;
(ii) adjusting pH of the culture broth after step (i) to pH 5-7;
(iii) fractionating a plurality of γ-PGA groups in the culture broth according to a distribution of γ-PGA molecular weight;
(iv) selecting a plurality of filtration membranes according to the molecular weight of the γ-PGA groups of step (iii);
(v) filtering the culture broth after step (ii) through the selected membranes of step (iii) in a sequence of molecular weight cutoff from high to low; and
(vi) recovering various γ-PGA products with different range of molecular weight.

5. The method of claim 4, wherein step (i) further comprises the steps of:
(1) adjusting pH of the culture broth below pH 2;
(2) centrifuging the culture broth after step (1) at 4° C. and 6,000-9,000 rpm for 30 minutes; and
(3) recovering the supernatant after centrifugation.

6. The method of claim 5, wherein the microorganism is Bacillus licheniformis.

7. The method of claim 4, wherein the selected membranes having molecular weight cutoff of 500 kD and 10 kD.

8. The method of claim 7, wherein the recovered γ-PGA products comprises a high molecular weight polymer (molecular weight being thousands of thousands) and a low molecular weight polymer (molecular weight being tens of thousands to hundreds of thousands).

9. The method of claim 4, further comprising a step of diluting the culture broth with water after step (ii).

10. The method of claim 9, the water volume for dilution is 3-5 fold based on the volume of culture broth.
